(19) United States
(12) Patent Application Publication
(10) Pub. No.: US 2010/0016730 A1
(43) Pub. Date: Jan. 21, 2010

(54) EXAMINATION SYSTEM, REHABILITATION SYSTEM, AND VISUAL INFORMATION DISPLAY SYSTEM

(75) Inventors: Toshiaki Tanaka, Hokkaido (JP); Hiroyuki Nara, Hokkaido (JP)

Correspondence Address: MOORE & VAN ALLEN PLLC, P.O. BOX 13706, Research Triangle Park, NC, 27709, US

(73) Assignee: SAPPORO MEDICAL UNIVERSITY, Hokkaido (JP)

(21) Appl. No.: 12/294,634

(22) PCT Filed: Oct. 3, 2006
(86) PCT No.: PCT/JP2006/320151

§ 371 Date: Sep. 26, 2008

(30) Foreign Application Priority Data
Mar. 30, 2006 (JP) 2006-094479

(51) Int. Cl. A61B 6/00

(52) U.S. Cl. 600/476

(57) ABSTRACT

In an examination system including a head mounted display having image pickup devices such as CCDs provided therein and a computer, coordinates of an image to be displayed in the head mounted display can be set object- or body-centered. An image captured by the image pickup device is supplied to the computer where it will be processed in a predetermined manner for display in the head mounted display.

### TECHNICAL FIELD DESCRIPTION

The present invention generally relates to an examination system, rehabilitation system and visual information display system, and more particularly, to an examination system, rehabilitation system and visual information display system, suitable for use to examine and rehabilitate a patient with a visual-spatial perception defect (visual-spatial agnosia) or visual defect as well as to help or assist such a patient in his or her daily life.

### BACKGROUND ART

The stroke likely to attack not a few high-aged persons and that will possibly remain as a sequela has been a serious problem over the world. In Europe and the United States, the measures against the stroke have been undergoing an accelerated comprehensive review since 1990s. In the Halsingborg Declaration in 1995 by the World Health Organization (WHO), it was stipulated as one of the aims to be achieved by 2005 that more than 70% of stroke patients shall be recovered in three months after being attacked by the stroke to such a level that they can live their daily life without help from others. In Japan, this aim has only been achieved to 60 to 70%. Of the stroke patients, 40% are bedridden elderly persons and 40% are elderly persons being under the home-visit nursing care service. According to the Heisei-4 (1992) Health and Welfare Statistics Directory (edited by the Statistics and Information Dept. of the Health and Welfare Minister's Secretariat and issued by the Health and Welfare Statistics Association on May 25, 1993), the total estimated number of patients is approximately 1,400,000. These patients were classified by accident and sickness. The majority of the total number of patients is shared by 210,000 cerebrovascular disease patients of which 14.8% are hospitalized and 24.9% are patients of more than 65 years in age. The rehabilitation normally takes a long time, and the mean number of days for which the cerebrovascular disease patients stay in hospital is 119 which is larger than the hospital days with other diseases. One of the causes for this long period of hospitalization is the visual-spatial perception defect. More specifically, the unilateral spatial neglect (USN) appears in 58% of the right brain hemorrhage group and 10% of the left brain hemorrhage group, and is seriously inhibiting the rehabilitation therapy.

The unilateral spatial neglect (USN) refers to a disorder in which the patient fails to respond or orient to stimuli present to the side opposite to a brain lesion. USN commonly causes the patient to behave abnormally in activities of daily living (ADL). That is, at an early stage of USN, the patient behaves abnormally as will be described below. He or she remains a neglect-side food uneaten at meals, is not aware of a care provider at the neglect side, bumps himself against a wall or door when walking or cannot let the arm through one of the sleeves of a clothes. Also, most of such patients commonly show disturbance of attention or low activation and overturn to bone fracture during the stay in hospital. Other central-nervous disorders (multiple sclerosis, tumor, trauma, cerebral paralysis, etc.) cause also a visual field defect such as unilateral or bilateral total blindness or hemianopia. Among these visual disturbances, the unilateral or bilateral total blindness or hemianopia and unilateral spatial neglect lead to walking difficulty and wheelchair-bound living. Thus, these visual disturbances lower the patient's ADL (Activities of Daily Living) and QOL (Quality of Life) and measures for solution of this problem should be taken urgently. However, extremely little research and development of welfare devices and equipment have ever been made for rehabilitation therapy of the patients with visual disturbances as complications of the central-nervous disorders as well as for functional compensation and improvement of the visual disturbances.

In these circumstances, the Inventors of the present invention have made research and development of a glass type virtual reality (VR) system with small 3CCD camera as a welfare device for use with vision-impaired persons (child) suffering from a central-nervous disorder and who cannot be treated. With this VR system, the Inventors have proposed to give visual information on a visual field defect region to one, free from the defect, or both eyes of such a patient in order to assure improved balance capacity, safe and stable walk, independent ADL and improved QDL (see “Relationship between visual function and falling in elderly person” by Toshiaki Tanaka, Physical Therapy 18 (9), 2001, pp. 847-851; T. Tanaka, S. Kojima, S. Shirogane, T. Ohyanagi, T. Izumi, H. Yumoto, S. Ino and T. Ifukube; 14th International Congress of the World Confederation for Physical Therapy (Proceedings, RP-P0-0982) 2003, Barcelona (Spain); Human Engineering, Vol. 41, No. 4 (2005), pp. 213-217). More specifically, an image of a scene or object, captured by the small 3CCD camera provided in a head mounted display (HMD) is corrected through processing by a computer and presented through HMD for viewing by a person having HMD mounted on the head. For augmenting the eyesight, for example, the image is enlarged about a gazed region, the image resolution is increased, the image contrast and coloration are sharpened and the image brightness is increased.

However, the method, the effectiveness, etc. of examining patients with visual-spatial perception defect such as the unilateral spatial neglect using the glass type VR system with the small 3CCD camera has not yet been clear. There are many problems to solve before the method is applied in practice.

Therefore, a subject to be solved by the present invention is to provide an examination system capable of examining easily, objectively, quantitatively and precisely what and how the disorder of an examinee suffering from a visual-spatial perception defect such as unilateral spatial neglect or a visual disturbance is.

Another subject to be solved by the present invention is to provide a rehabilitation system capable of giving an examinee with a visual-spatial perception defect such as unilateral spatial neglect or a visual disturbance an appropriate training depending on what and how his or her disorder is and performing effective rehabilitation therapy, help/assistance, etc.

Further subject to be solved by the present invention is to provide a visual information display system capable of giving visual aid and assistance to an examinee with a visual-spatial perception defect such as unilateral spatial neglect or a visual disturbance as well as to ordinary normal people to assure improved activities of daily living and quality of life.

### DISCLOSURE OF THE INVENTION

The Inventors of the present invention have devoted themselves to research and development for solving the above-mentioned subjects and found that for examining what and how the disorder of patients suffering from a visual-spatial perception defect such as unilateral spatial neglect or a visual disturbance is, training such patients or improving their activities of daily living and quality of life, it is effective for the purpose of elaborate evaluation of the visual-spatial perception defect to adapt the system so that the coordinates in which a scene or object image picked up by a small 3CCD camera or the like provided in the head mounted display is to be displayed can be set object- or body-centered, instead of simply displaying the image corrected through processing by a computer to the patient via the head mounted display. Based on the above findings, the Inventors worked out the present invention.

To solve the above-mentioned subject, according to the first embodiment, there is provided an examination system comprising a head mounted display in which one or more image pickup devices are provided,

coordinates in which an image is to be displayed in the head mounted display being set object- or body-centered.

Also, according to the second embodiment, there is provided a rehabilitation system comprising a head mounted display in which one or more image pickup devices are provided, coordinates in which an image is to be displayed in the head mounted display being set object- or body-centered.

Also, according to the third embodiment, there is provided a visual information display system comprising a head mounted display in which one or more image pickup devices are provided,

coordinates in which an image is to be displayed in the head mounted display being set object- or body-centered.

In the first to third embodiments, the object-centered allocentric coordinates define a framework structured for each of individual spatial objects. In case the spatial objects are symmetric with respect to a certain axis, a reference frame can be structured based on the axis. However, since all the spatial objects are not symmetrical, the reference frame in the center of symmetry varies depending upon how the observer recognizes it and also upon a given task. On the other hand, the body-centered (egocentric) coordinates (head/body coordinates) define a spatially symmetrical framework in which the coordinates are centered to the observer's body (or a part of the body). The human body is coded in position and direction different from those of the spatial object depending upon a plurality of moving parts such as the eyes, neck, body, etc., which is characterized by the fact that the direction and position of a spatial object vary in the reference frames as the observer moves.

The image pickup device provided in the head mounted display should be a CCD, and more preferably a 3CCD that can produce a color image (three CCDs for blue, green and red, respectively). The number of pixels of the CCD should be at least 200,000 but may be larger. The image pickup device may be other than CCD, for example, a MOS or CMOS imaging device. It should preferably be a type capable of producing a color image. The image pickup device is normally designed in the form of a small camera. It is mounted on the top of the head mounted display and in a position where it faces away from the eye of an examinee or user when mounted on him or her and the optical position of the examinee's or user's eye coincide with that of the image pickup device. The head mounted display is adapted, as necessary, to measure the movement of the examinee's or user's head. More specifically, the head mounted display includes a unit for measurement of the head movement.

The head mounted display may be of a completely shielded type. However, it should preferably be of an open type permitting the wearer of the head mounted display to have a peripheral vision closely related to the sense of equilibrium in order to prevent a virtual sick-feeling from being caused by a large abrupt movement of the examinee's or user's head on which the head mounted display is put. Also, it should be adapted to present a stimuli for apparent motion. More specifically, a horizontal line as a reference and another horizontal line which moves in a direction opposite to a head movement are displayed to present such an apparent-motion stimuli.

Typically, the above examination, rehabilitation and visual information display systems further comprise a processing/control unit that processes a image captured by the image pickup device for display in the head mounted display. The processing/control unit is normally a computer.

To set the coordinates for an image to be displayed in the head mounted display to object- or body-centered coordinates, an image captured by the image pickup device may be supplied to the computer where it will be processed to produce an image for display in the head mounted display or an image captured by a digital camera or the like may be superposed on an image captured by the image pickup device for display in the head mounted display, for example. Signals may be transferred between the computer that processes and control an image captured by the image pickup device and the head mounted display via a cable or by radio communication.

How an image captured by the image pickup device is to be processed is appropriately determined based on how a patient with a visual-spatial perception defect such as unilateral spatial neglect and visually impaired humans are to be examined, rehabilitated, helped, assisted or otherwise cared. Specifically, the image is processed as follows. Namely, a real-space image captured by the image pickup devices is subjected to edge enhancement, reduction, enlargement, addition of attention information, color transform, binarization, etc. For example, combining attention information with a real-space image assists a USN patient in recognizing a neglect space in the real space, and reconstructing visual information in a space excluding a neglect space assists the patient in recognizing the space. Also, image processing such as enlargement, edge enhancement, color transform or binarization permits to present the patient easy-to-recognize optimum visual information. It should be noted that the sight change by the enlargement or reduction should preferably be made without variation of the center of real space.

For example, an examination or training using a test sheet or the like can be effected with the image pickup devices or small camera in the head mounted display being kept focused on the test sheet or the like without being influenced by any position of the examinee or user. As one example of such applications, the HMD system is so adapted the test sheet or the like is imaged by a digital camera or the like fixed to a chair on which the examinee or user going to undergo an examination or training is sitting and the image is formed for display in the head mounted display. And the system is so adapted that the image of the test sheet or the like captured by the digital camera or the like is subjected to enlargement or reduction for display in the head mounted display.

### BRIEF DESCRIPTION OF THE DRAWINGS

FIG. 1 schematically illustrates a visual information display system according to one embodiment of the present invention.

FIG. 2 schematically illustrates a head mounted display included in the visual information display system according to the one embodiment of the present invention.

FIGS. 3A and 3B schematically illustrate first examples of an image displayed on the head mounted display in the visual information display system according to the one embodiment of the present invention.

FIGS. 4A and 4B schematically illustrate second examples of an image displayed on the head mounted display in the visual information display system according to the one embodiment of the present invention.

FIGS. 5A and 5B schematically illustrate third examples of an image displayed on the head mounted display in the visual information display system according to the one embodiment of the present invention.

FIGS. 6A and 6B schematically illustrate fourth examples of an image displayed on the head mounted display in the visual information display system according to the one embodiment of the present invention.

FIGS. 7A and 7B schematically illustrate fifth examples of an image displayed on the head mounted display in the visual information display system according to the one embodiment of the present invention.

FIG. 8 schematically illustrates a result of a line cancellation test made with the use of the visual information display system according to the one embodiment of the present invention as an examination system.

FIG. 9 schematically illustrates a result of a star cancellation test made with the use of the visual information display system according to the one embodiment of the present invention as an examination system.

FIG. 10 schematically illustrates a measuring system for a unilateral spatial neglect test in which the visual information display system according to the one embodiment of the present invention is used as an examination system.

FIG. 11 schematically illustrates a unilateral spatial neglect testing method in which the visual information display system according to the one embodiment of the present invention is used as an examination system.

### BEST MODE FOR CARRYING OUT THE INVENTION

The present invention will be described in detail below concerning the one embodiment of the present invention with reference to the accompanying drawings.

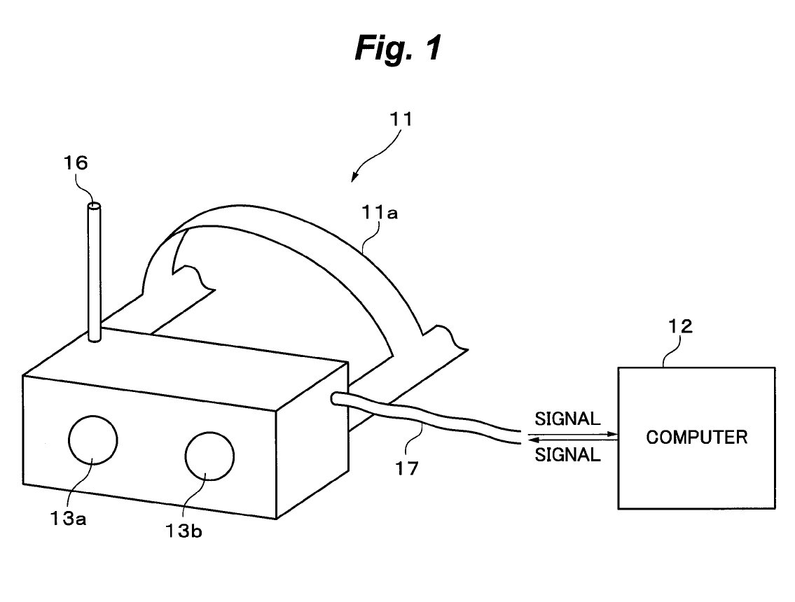

FIG. 1 shows the visual information display system according to the one embodiment of the present invention. The visual information display system can be used as an examination system or a rehabilitation system for handicapped persons such as visually impaired humans.

As shown in FIG. 1, the visual information display system includes a head mounted display 11 and computer 12.

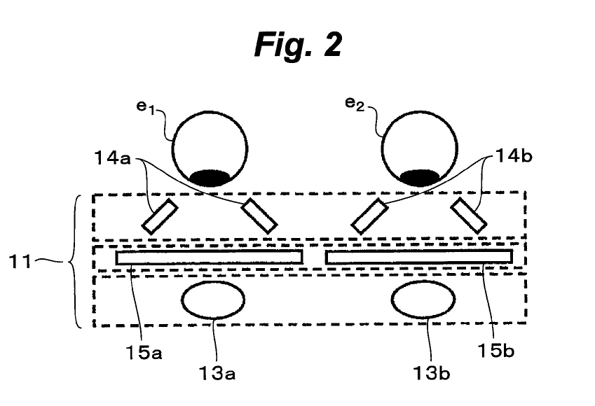

The head mounted display 11 is constructed as shown in FIG. 2. As shown in FIG. 2, the head mounted display 11 includes two 3CCD cameras 13a and 13b provided on the front face thereof, more specifically, in positions where they will optically correspond to both eyes e₁ and e₂ when the head mounted display 11 is mounted on the head of a user or examinee, two eye cameras 14a and 14b provided in positions corresponding to the both eyes e₁ and e₂, and two display units 15a and 15b provided in positions optically corresponding to the both eyes e₁ and e₂ and each of which is a liquid crystal display (LCD) or a display of any other type.

As shown in FIG. 1, the head mounted display 11 also includes a head/body motion measuring unit 16 incorporating a GPS antenna and receiver (radio LAN receiver), not shown. Further, the head mounted display 11 has a band 11a with which the head mounted display 11 is to be fixed on the user's or examinee's head.

As will be known from FIG. 1, a cable 17 is connected between the head mounted display 11 and computer 12 for mutual transmission of signals. However, it should be noted that signals may be transmitted by air between the head mounted display 11 and computer 12. Images captured by the 3CCD cameras 13a and 13b of the head mounted display 11 are supplied to a processing/control unit in the body of the computer 12. The images are processed in a predetermined manner in the processing/control unit. The images thus processed are displayed on display units 15a and 15b for viewing by the user or examinee. In contrast, movement of the eyes e₁ and e₂ of the user or examinee is measured by the eye cameras 14a and 14b and the results of the eye movement are also supplied to the processing/control unit. The results of the eye movement will thus reflect the predetermined processing, in the processing/control unit, of the images captured by the 3CCD cameras 13a and 13b.

The computer 12 normally includes a computer body (processing/control unit) and display. It should be noted that the computer 12 may be either a desk-top type or a notebook type. The processing/control unit includes a processor, main memory, auxiliary memory, etc. The auxiliary memory may be of any type such as hard disk drive or the like. Also, the auxiliary memory may be built in the processing/control unit or provided outside the computer body. The auxiliary memory has stored therein a predetermined program written using a predetermined programming language. For execution of the program, it is read out of the auxiliary memory into the main one or it is downloaded via wireless LAN or the like. The program is executed by the processor or by a server connected via a network. It should also be noted that the processing/control unit may have connected thereto an input unit such as a keyboard or mouse, and other peripheral unit, which are not illustrated.

In this embodiment, it is possible by designing the program using the predetermined programming language to set object- or body-centered coordinates in which images are to be displayed on the screens of the display units 15a and 15b of the head mounted display 11. This coordinate setting can easily be made by the user or examinee by operating the keyboard, mouse or the like of the computer.

The processing/control unit can make any one of various processes selected as necessary, of which concrete examples will be explained below:

(1) Edge-Enhancing Display

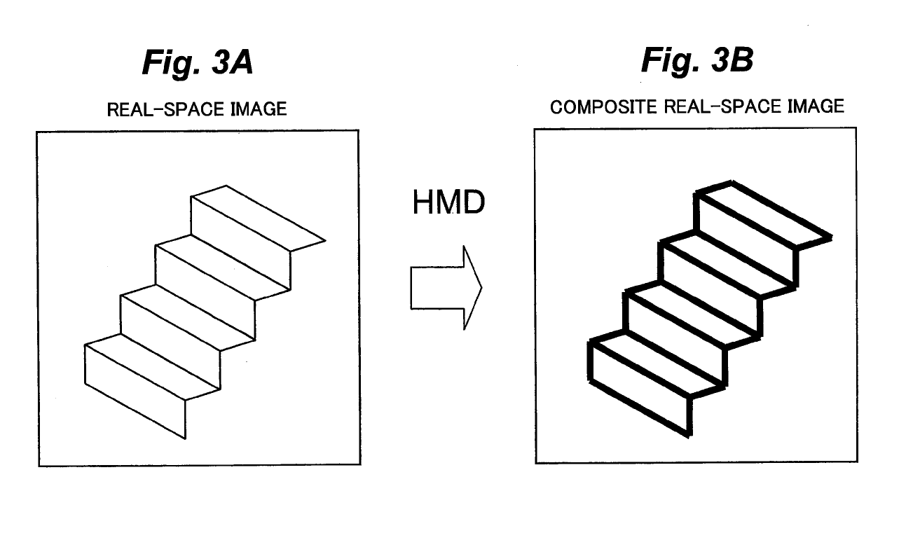

Edge of a real-space image is enhanced for easier viewing. For example, a real-space image of stairs as shown in FIG. 3A is processed by the processing/control unit to provide a composite real-space image (image to be displayed on the display units 15a and 15b of the head mounted display 11) in which the image edge of the real-space image is enhanced as shown in FIG. 3B.

(2) Reducing Display

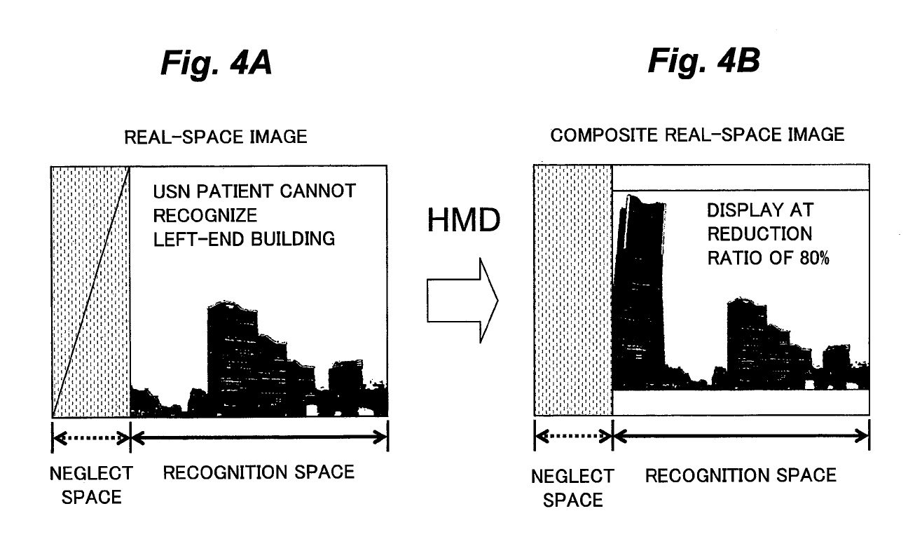

Visual information on a neglect space is also displayed in a recognition space of a real-space image for easier recognition of visual information on the neglect space. For example, in case a unilateral-spatial neglect patient cannot recognize a building at the left end of a neglect space in a real-space image of buildings as shown in FIG. 4A, the real-space image is horizontally reduced in size in a composite real-space image as shown in FIG. 4B, whereby the building in the neglect space can be displayed in the recognition space so that the unilateral-space neglect patient can also recognize the building. In this example, the reduction ratio is 80%. Generally, however, the reduction ratio should preferably be over 70% and under 90% (see “Human Engineering”, Vol. 41, No. 4 ('05), pp. 213-217). Thus, an easier-to-recognize space can be displayed by gradually compressing the field from the peripheral field toward the center without changing the center of the real space in the field transformation by such reduction.

(3) Attention Information Display

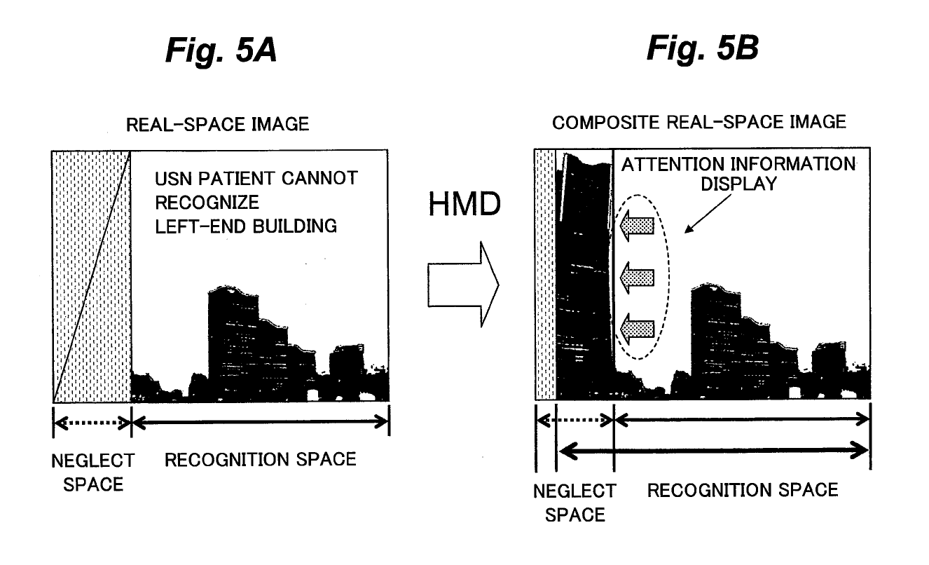

Attention information on a recognition space is also displayed in a real-space image for recognition of visual information on a neglect space. For example, in case a unilateral-spatial neglect patient cannot recognize a building at the left end of a neglect space in a real-space image of buildings as shown in FIG. 5A, attention information is displayed at the left end of the neglect space in the recognition space in a composite real-space image as shown in FIG. 5B, whereby it is possible to recognize visual information on the neglect space as well. In this example, arrows are used as the attention information. Actually, virtual-space image including predetermined attention information is generated in position, and the virtual-space image is combined with a real-space image to provide a composite real-space image.

(4) Enlarging Display

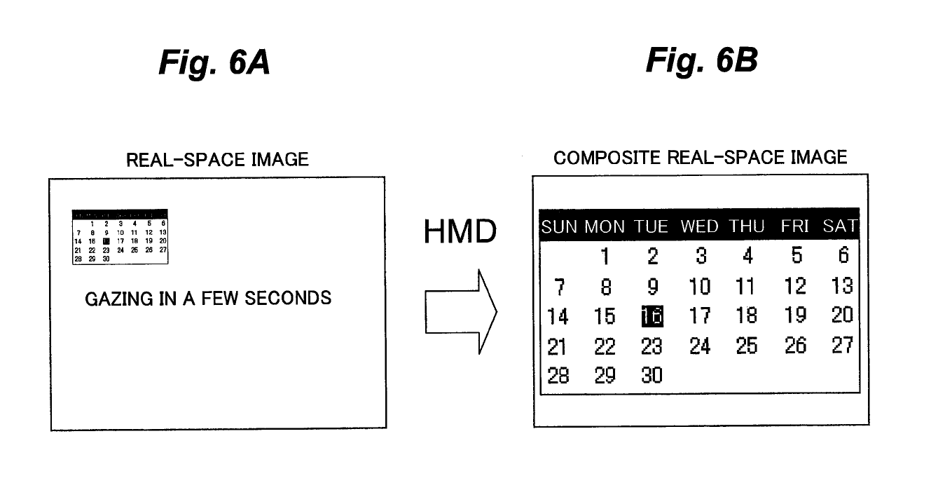

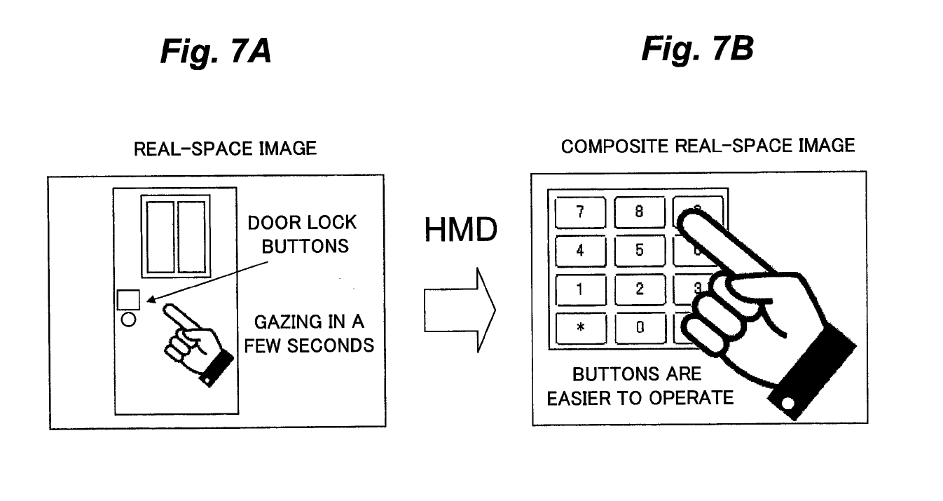

A situation in which the examinee has gazed an object in a real space for a few seconds is detected on the basis of eye movement and what the examinee has gazed during the detection is made more recognizable by displaying it in a larger scale in a composite real-space image. For example, when the examinee gazes a calendar in a far position as in a real space as shown in FIG. 6A, the calendar is displayed in a larger scale in a composite real-space image as shown in FIG. 6B. Thus, the calendar is easily viewable. Also, when the examinee gazes door lock buttons for a few seconds in a real space as shown in FIG. 7A, the door lock buttons are displayed in a larger scale in a composite real-space image as shown in FIG. 7B. Thus the door lock buttons are easily operable.

The above operations and display can automatically be effected in a manner suitable for each user by the processing/control unit in the computer 12 on the basis of results of examinations previously made using the visual information display system, data derived from eye movement measured by the eye cameras 14a and 14b, data derived from the head and body movement measured by the head/body movement measuring unit 16, and others.

The visual information display system is used as an examination system in unilateral spatial neglect (USN) tests for example as will be explained below.

1. Subjects

Subjects in this study were eight patients who had suffered from a stroke (of 67.1 years in mean age) and from whom informed consent were obtained. The patients were examined by two therapists by a test on ADL and found to show a neglect. All the patients were further examined by two medical doctors using CT (Computed Tomography) or MRI (Magnetic Resonance Imaging) and found to have a right hemisphere damaged. The patients did not include any patients with weak visual acuity, dementia, hemianopsia, apraxia or those being left-handed. The patients were those who could sit on an ordinary chair by themselves. The examinations were made in 4 to 27 weeks from the appearance of disease. The patients thus examined have characteristics as collectively shown in Table 1.

TABLE 1
CHARACTERISTICS OF PATIENTS

| PATIENT | AGE | | | TIME OF REHABILITATION | FIM-M |
|---|---|---|---|---|---|
| NO. | (YEARS) | DIAGNOSIS | LESION* | ONSET (WEEKS) | SCORE |
| 1 | 75 | I | FTP | 6 | 30 |
| 2 | 65 | I | BgFPT | 1 | 38 |
| 3 | 64 | H | Th | 1 | 61 |
| 4 | 63 | H | Bg | 1 | 35 |
| 5 | 56 | I | PT | 1 | 85 |
| 6 | 70 | I | Bg | 1 | 33 |
| 7 | 79 | I | FPT | 1 | 86 |
| 8 | 68 | I | BgFPT | 2 | 72 |

ABBREVIATIONS: I: INFRACTION, H: HEMORRHAGE, F: FRONTAL LOBE, P: PARIETAL LOBE, T: TEMPORAL LOBE, Bg: BASAL GANGLIA, Th: THALAMUS
*ALL LESIONS WERE RIGHT SIDED

2. Functional Assessment

The Functional Independence Measure (FIM) was executed as an ADL evaluation. The FIM motor sub scores (FIM-M) were used for measure of disability as the best predictors of rehabilitation length of stay for stroke. Moreover, the two therapists evaluated the patients who exhibited specific neglect behaviors in ADL using a special checklist shown in Table 2. The checklist used a modified version of Halligan's checklist.

TABLE 2
CHECKLIST OF EVERYDAY NEGLECT BEHAVIORS

| | |
|---|---|
| 1. | DOES THE PATIENT SHOW DIFFICULTIES WHEN: TALKING OR COMMUNICATING WITH OTHERS? |
| 2. | DOES THE PATIENT NEGLECT THE LEFT/RIGHT SIDE OF PERSONAL SPACE? |
| 3. | DOES THE PATIENT SHOW DIFFICULTIES IN EATING? |
| 4. | DOES THE PATIENT SHOW DIFFICULTIES IN GROOMING (SELF-CARE SKILLS, WASHING, |
| | BATHING, ETC.)? |
| 5. | DOES THE PATIENT SHOW DIFFICULTIES IN DRESSING? |
| 6. | DOES THE PATIENT SHOW DIFFICULTIES IN TRANSFERRING (FROM A BED |
| | TO W/C, ETC.)? |
| 7. | DOES THE PATIENT SHOW DIFFICULTIES IN LOCOMOTION 1 (THE PATIENT COLLIDES AGAINST |
| | OBJECTS AND WALL ON THE AFFECTED SIDE. THE PATIENT CAN NOT NEGOTIATE A W/C BETWEEN |
| | DOORS, CURVES, ETC.)? |
| 8. | DOES THE PATIENT SHOW DIFFICULTIES IN LOCOMOTION 2 (THE PATIENT TURNS TOWARD THE |
| | DIRECTION OF THE AFFECTED SIDE.)? |
| 9. | DOES THE PATIENT SHOW DIFFICULTIES DURING PT EXERCISE? |
| 10. | DOES THE PATIENT SHOW DIFFICULTIES DURING OT EXERCISE? |

3-1 Evaluation for USN

3-1-1. Common Clinical Test

To assess neglect, the line and star cancellation tests as included in the Behavioral Inattention Test (BIT) were made on the subjects. We used the BIT devised by Wilson et al. and modified in a Japanese version by Ishiai et al for use with the Japanese people.

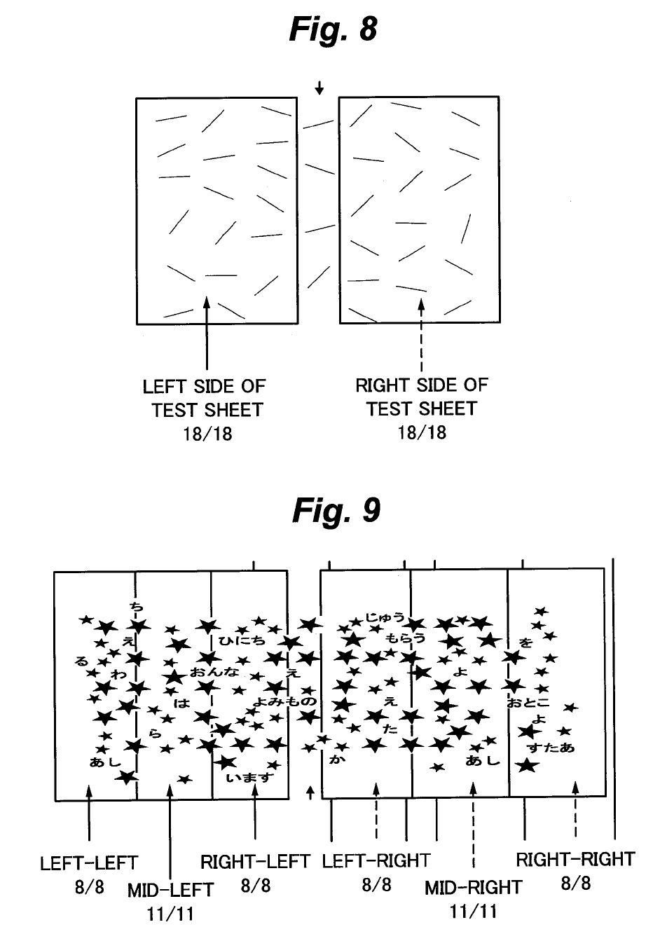

For the line cancellation test (count of cancellations: minimum 0 to maximum 36), the subjects were given each a single sheet of paper on which 6 lines different in direction from each other were drawn vertically with 18 such lines being drawn on either side of the sheet as shown in FIG. 8. The subjects were instructed to cancel all the lines. When a subject fails to mark more lines on the left side than on the right side, he or she is considered to exhibit left-sided neglect. Degree of such neglect was assessed based on a proportion in number between omitted lines and all the lines marked on the test sheet. The line cancellation test sheet was divided into right half and left half and the correctly canceled lines were counted first in the right half and then in left half of the sheet. In this test, a count of 34 lines correctly canceled was used as a cutoff value. For the star cancellation test (count of star cancellations: minimum 0 to maximum 54), there was prepared an A4-sized stimulus sheet having provided thereon 56 targets (smaller stars) pseudo-randomly interspersed with distracter items as shown in FIG. 9. The total number of marked small stars was recorded. Actually, the targets are laid in six columns with two additional targets being provided in the central area not belonging to the six columns. The examiner first explained the entire sheet and requested the subjects to mark the two targets located in the central area by way of example. However, this marking was not counted. Next, the examiner asked the subjects to cancel the remaining small stars. The number of the targets omitted in each horizontal half of the test sheet was counted. The star cancellation test sheet had six areas (left-left, middle-left, right-left areas and right-right, middle-right, left-right areas). It was determined how many stars had been canceled correctly in the six areas. A count of 51 stars correctly canceled was taken as a cutoff value.

3-2. Special Test with HMD

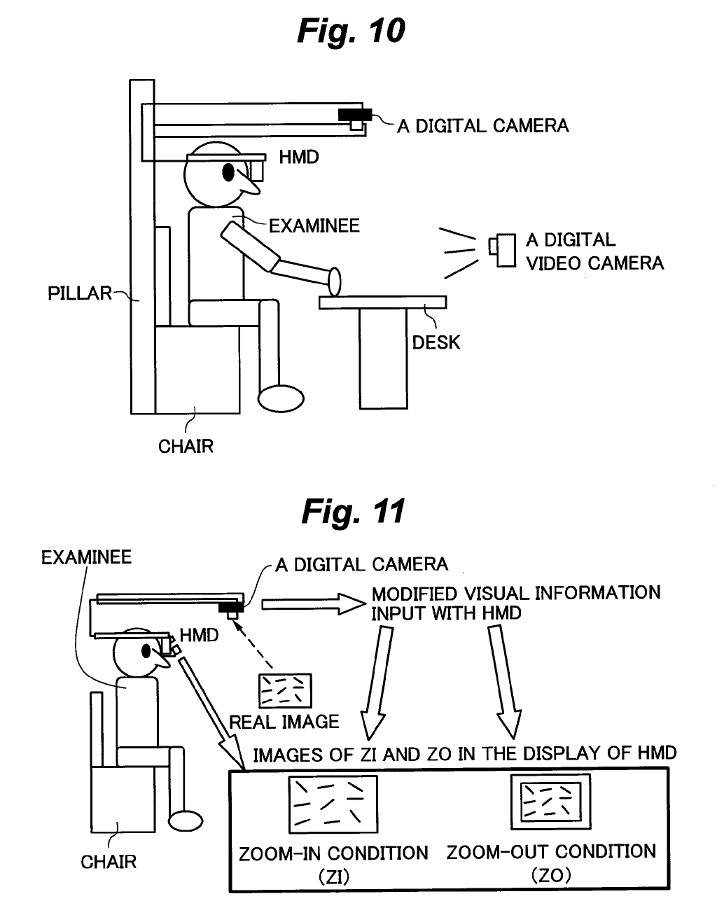

(a) Experimental Apparatus (Also See FIG. 10)

The main experimental apparatus includes a digital camera, HMD (GT270 from Canon Inc.), and a digital video camera. HMD is a glass type display including two TFT liquid crystal panels (270,000 pixels, 99.9% in number of effective pixel and a mass of 150 g). The digital camera is used to take a picture of a test sheet placed on a desk, and HMD projects the image captured by the digital camera onto the retina of the subject. Moreover, the digital video camera was used to record subject's head movement for qualitative analysis.

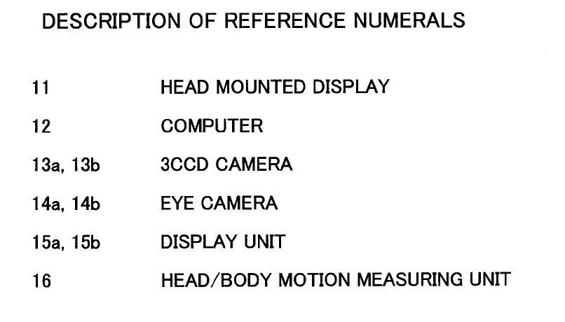

(b) Assessments of USN Using HMD (Also See FIG. 11)

The Inventors attempted to determine how much USN alters when the coordinates of the subject's visual field was set object-centered by HMD. To this end, the two different types of lenses were used in the digital camera to change visual field and the test sheet was displayed to the examinee by HMD for two special tests as will be described below:

1) Special Test 1:
With zoom-in (ZI) in which only the image of the test sheet can be displayed with the combination of HMD and digital video camera.

2) Special Test 2:
With zoom-out (ZO) in which the test sheet image can be displayed in a size of 7/10 of that of the image in the special test 1 by changing the lens from one to another.

3-3. Experimental Procedure

The subjects was seated on a wheelchair or a chair whose back is upright as a starting point, as necessary. The test sheet placed on the desk was positioned on a body midline of each subject. All tests were done with no time restriction.

The subjects were first subjected to a normal test without HMD as a common clinical test, and then to two special tests with HMD (with ZI and ZO). The results of the line cancellation test were scored based on how many lines had been canceled correctly and then the score was rated for the right and left halves, respectively, of the test sheet. The results of the star cancellation test were scored based on the number of correctly canceled targets for each of the six areas (left-left, middle-left, right-left areas and right-right, middle-right, left-right areas) of the test sheet. All the subjects were subjected in random order to common clinical test and the two special tests (with ZI, ZO). The examiner made sure that the image on the HMD monitor was from the digital camera. Moreover, the movement of head, body and upper/lower extremities was qualitatively analyzed during these tests for finding an abnormal movement.

4. Data Analysis

SPSS statistical software (7.5.2J) was used for all statistics in this study. An ANOVA or Student's t test was used for comparison between the results of the common clinical test and two special tests using HMD. Moreover, the Student's t or ANOVA test was used for comparison in each of the line and star cancellation tests. Multivariate ANOVA tests were performed in each group, and when significant differences were found at the 5% significance level, Sheffe post hoc tests were effected.

Also, the digital video camera was used for qualitative analysis in a sagittal or frontal plane of the movement of the head, body and upper/lower extremities during all the tests.

<Results>

In this embodiment, the mean value of FIM-M of all the subjects was 53.0±21.6 points (see Table 1). The subjects were considered to need maximal or moderate assistance for some motion in ADL.

As a common clinical test on USN, the first evaluation of the frequency of presence of neglect in ADL proved that 75% of all the subjects exhibited a USN symptom in dressed activities (see Table 3). For example, patients with USN cannot easily put on their clothes at their left side. Moreover, 62.5% of the subjects exhibited a USN symptom in activities such as transfer and relocation (see Table 3). As found in the head-motion analysis in the common clinical test, the subjects began searching from the right side in both the line and the star cancellation tests. In the normal performance, the head naturally turned from the right to left as it moved in the line cancellation test. However, the head movement to the left was insufficient for searching from the right side in the both tests. For the line cancellation test under the common condition, the mean percentage of the correct cancellation at the left-hand area in the test paper was 94.4%. The mean percentage of the correct cancellation in the right-hand area was 100 %. None of the subjects showed any such percentage below the cutoff value (see Table 4). As the result of the star cancellation test in the common clinical test, the mean percentage of the correct cancellation in the left-left area was 91.1% (see Table 5). The mean percentage of the correct cancellation in the middle-left area was 89.2% and that in the right-left side was 84.4%. The mean percentage of the correct cancellation in the right-right area was 92.9%, that in the middle-right area was 96.4%, and that in the left-right area was 81.8%. Three of the subjects showed a mean percentage of the correct cancellation below the cutoff value. They were considered to be abnormal.

TABLE 3
RATIO OF USN SYMPTOMS IN ADL AND EXERCISE

| | n = 8 | RATIO OF USN (%) |
|---|---|---|
| TALKING OR COMMUNICATING WITH OTHERS | 4 | 50.0 |
| MOTION ON BED (NEGLECTING THE LEFT SIDE, ETC.) | 2 | 25.0 |
| EATING | 1 | 12.5 |
| GROOMING (SELF-CARE SKILLS, WASHING, BATHING, ETC.) | 2 | 25.0 |
| DRESSING | 6 | 75.0 |
| TRANSFERRING (FROM A BED TO W/C, ETC.) | 5 | 62.5 |
| LOCOMOTION 1 (THE PATIENT CANNOT NEGOTIATE A W/C | 5 | 62.5 |
| BETWEEN DOORS, CURVES, ETC.) | | |
| LOCOMOTION 2 (THE PATIENT TURNS TOWARD THE DIRECTION | 5 | 62.5 |
| OF THE AFFECTED SIDE.) | | |
| NEGLECT DURING PT EXERCISE | 6 | 75.0 |
| NEGLECT DURING OT EXERCISE | 7 | 87.5 |

TABLE 4
MEAN PERCENTAGE OF CORRECT ANSWERS OF THE LINE
CANCELLATION TEST IN THE COMMON BIT TEST.

| | MEAN PERCENTAGE OF |
|---|---|
| | CORRECT ANSWERS (%) |
| LEFT SIDE OF TEST SHEET | 95.1 ± 13.8 |
| RIGHT SIDE OF TEST SHEET | 100 ± 0 |

TABLE 5
MEAN PERCENTAGE OF CORRECT ANSWERS OF THE STAR
CANCELLATION TEST IN THE COMMON BIT TEST

| | PERCENTAGE OF |
|---|---|
| | CORRECT ANSWERS (%) |
| LEFT-LEFT AREA | 91.1 ± 13.7 |
| RIGHT-LEFT AREA | 81.8 ± 31.1 |
| MID-LEFT AREA | 89.3 ± 8.6 |
| MID-RIGHT AREA | 96.4 ± 5.9 |
| LEFT-RIGHT AREA | 84.4 ± 30.1 |
| RIGHT-RIGHT AREA | 92.9 ± 14.0 |

As known from the motion analysis of head motion in the special test with HMD, the subjects were began searching from the right side in both the line and the star cancellation tests. However, seven of all the subjects kept turning to the right half of the test sheet alone. They did not turn to the left half. As the result of the line cancellation test under the ZI condition in the special test with HMD, the mean percentage of the correct cancellation in the left half of the test sheet was 61.8% (see Table 6). The means percentage of the correct cancellation in the right half was 92.4%. Under the ZO condition, the mean percentage of the correct cancellation in the left half of the test sheet was 79.9%. The mean percentage of the correct cancellation in the right half was 91.7%. Under either of the ZI and ZO conditions, the score of correct cancellation in the left half was significantly greater than that in the right half (p<0.05). There was a significant difference between the common clinical test and ZI conditions of the special test with HMD for the left side score (p<0.05). As the results of the star cancellation test under the ZI condition in the special test with HMD, the mean percentage of the correct cancellation in the left-left area was 60.7% (see Table 7.) The mean percentage of the correct cancellation in the middle-left area was 69.6%, and the mean percentage in the right-left area was77.9%. The mean percentage of the correct cancellation in the right-right area was 87.5%, that in the middle-right area was 92.9%, and that in the left-right area was 87.0%. Under the ZO condition, the mean percentage of the correct cancellation in the left-left area was 69.7%. The mean percentage of the correct cancellation in the middle-left area was 70.8% and that in the right-left area was 77.9%. The mean percentage of the correct cancellation in the right-right area was 97.9%, that in the middle-right area was 87.5%, and that in the left-right area was 92.4%.

TABLE 6
MEAN PERCENTAGE OF CORRECT ANSWERS
OF THE CANCELLATION TEST IN DIFFERENT
THREE CONDITIONS

| | CORRECT ANSWERS | CORRECT ANSWERS |
|---|---|---|
| | FOR LEFT SIDE (%) | FOR RIGHT SIDE (%) |
| COMMON TEST | 95.1 ± 13.8^{ab} | 100 ± 0 |
| ZI CONDITION | 61.8 ± 34.3^{a} | 92.3 ± 11.1 |
| ZO CONDITION | 79.8 ± 37.6^{a} | 91.7 ± 14.5 |

^{a}SIGNIFICANT DIFFERENCE BETWEEN RIGHT AND LEFT (p < 0.05)
^{b}SIGNIFICANT DIFFERENCE BETWEEN COMMON TEST AND ZI (p < 0.05)

TABLE 7
MEAN PERCENTAGE OF CORRECT ANSWERS OF THE STAR
CANCELLATION TEST IN THREE DIFFERENT CONDITIONS
MEAN PERCENTAGE OF CORRECT ANSWERS (%)

| | COMMON | ZI | ZO |
|---|---|---|---|
| | TEST | CONDITION | CONDITION |
| LEFT-LEFT AREA | 91.1 ± 13.7 | 60.7 ± 47.0 | 66.7 ± 51.6 |
| RIGHT-LEFT AREA | 81.8 ± 31.1 | 87.0 ± 10.2 | 69.7 ± 38.4 |
| MID-LEFT AREA | 89.3 ± 8.6 | 69.6 ± 37.4 | 70.8 ± 42.3 |
| MID-RIGHT AREA | 96.4 ± 5.9 | 92.9 ± 6.4 | 87.5 ± 13.7 |
| LEFT-RIGHT AREA | 84.4 ± 30.1 | 77.9 ± 37.0 | 69.9 ± 38.4 |
| RIGHT-RIGHT AREA | 92.9 ± 14.0 | 87.5 ± 14.3 | 97.9 ± 4.9 |

### <Discussion>

All the subjects reported that the HMD displayed a brighter, clearer image at a nearly real time and HMD put on them gave no discomfort to them. In this embodiment, HMD can provide an image as if the subject were watching a 52-inch display screen 2 m away from him or her. Moreover, the indirect visual field can be changed in range by adapting the HMD system so that the computer controls the selection of an input method for use of HMD.

A digital camera was used for projecting an image of the test sheet onto the liquid crystal display screen of the HMD. This digital camera was fixed so that the test sheet image on the liquid crystal screen of HMD would not move even if the head moved during a test. This implies that in the special test with HMD, more appropriate object-centered allocentric coordinates could be produced than in the common condition test. In this embodiment, the ZI condition was the same as that in the object-centered allocentric coordinates.

The results of the motion analysis made during the special test with HMD showed that the subjects had a tendency to have a view mainly on the right side of the test sheet under the conditions of ZI and ZO in contrast to the behavior found in the common clinical USN examination. In video recording for qualitative motion analysis, the subject showed a tendency to have an intensive view of the right side of the test sheet when subjected to the special test with HMD. It is possible that the subject's neglect was enhanced by HMD. Since in the special test with HMD, there were the object-centered allocentric coordinates, the subject was focused more on the test sheet than in the common clinical test. This means that if the subject pays too much attention to an object, it will be possible that he or she ignores the left side of the test sheet. Moreover, Ishiai et al. examined USN patient's eye movement using an eye camera. The results of examination showed that the healthy persons and patients having no USN symptoms but homonymous hemianopia could move their eyes while directing them toward the center of the test sheet. However, the patients with homonymous hemianopia and also USN symptoms veered to the right side and could not direct their eyes to the right side. Since HMD allows the patient to stay focused on the object (test sheet) by limiting the view area more than in the common clinical test, it can clarify the left neglect area more.

The correct cancellation in the left space under the ZI and ZO conditions was significantly lower than that in the common clinical test. Moreover, the correct cancellation under the ZO condition was slightly greater than that under the ZI condition. It may be considered that the ZI condition permitted the patient to stay focused more on an object than the ZO condition. These results indicated that the patients with USN stay focused on an object would have the USN symptoms aggravated more. The check list prepared by Halligan et al. shows patients include many whose behaviors of dressing, transfer and relocation are highly impaired by USN. With the common BIT, there could not satisfactorily be revealed USN in which the correct cancellation in the left space was higher than 80%. With the special test with HMD, however, there could be revealed USN in which the correct cancellation in the left space was about 60%. Namely, the examination by HMD enables easier detection of USN symptoms which could not be easily found by the common clinical test.

In the early research by the Inventors, use of HMD improved the neglect symptoms in all subjects who had right cerebral hemisphere damage. Rossetti et al. investigated the effect of the application of a prism to neglect symptoms in which the subject's visual field was pathologically shifted from the midline to the right. They reported that all patients having the visual field optically shifted to the right were recovered from the disease owing to the manual display of body and midline to them and classical neuropsychological tests made on them. Also, Lee et al., Woo and Mandelmant suggested that a Fresnel prism could effectively be used on a spectacle lens for recovery from various visual-field defects. The recovery achieved using HMD means that HMD gives the brain a signal that stimulates the natural recovery as in the prism adaptation. Further, the HMD system provides more effective recovery from a left neglect than the Fresnel prism placed on the spectacle lens. However, since a high power Fresnel prism membrane for a wide field of view is not clear, the prism caused a real image to be distorted, which led to lowered capabilities of visual acuity. In contrast, HMD is capable of providing various fields of view without deterioration of visual acuity.

The HMD system is advantageously non-invasive and safe and permits to easily change the size of a visual field. The standard clinical examinations are used mainly in a horizontal two-dimensional plane, but the HMD system permits clinical examinations related more closely to ADL in other planes, frontal or sagittal. On the other hand, the HMD system should be designed for a higher portability, reduced weight and a decreased delay of response between the computer and HMD for data transformation. The delay time of this HMD system is 50 msec. Therefore, the HMD system should adopt a higher-level technology of processing, recording and displaying a changed visual field of view that changes in real or near-real time.

The HMD system may be used as an evaluation device to play an important role in the neuropsychological rehabilitation of unilateral spatial neglect. Bowen et al. made a systematic review of published reports. According to their report, right brain damage (RBD) and left brain damage (LBD) were compared directly with each other in seventeen of the reports and it is apparently supported by the systematic review of data set forth in the reports that USN occurs more commonly after RBD rather than after LBD. However, there were not accurately estimated occurrence of USN after a stroke and recovery from USN. They suggested that different USN disorders exist each type of which needs a peculiar rehabilitation approach for rehabilitation. The system is adapted for selection of various visual-field inputs for accommodation of different USN degrees of the patients, and hence defines a clinical meaning for new assessment. For clinical assessment of USN, various images in HMD can be processed for color change, partial enlargement or reduction of a real image by the computer. Namely, appropriate visual information can be produced in HMD for each USN patient.

In this embodiment, an image displayed in HMD can be evaluated in object-centered allocentric coordinates. That is, the HMD system can be used with focus on the image evaluation in the object-centered allocentric coordinates rather than in egocentric (body-centered) coordinates. Of course, HMD may also be adapted to produce body-centered (egocentric) coordinates for evaluation of an image. In this case, HMD display should be synchronized with a small CCD camera which is to be placed on the head or body. Moreover, eye and head movement should be measured for analysis of the eye-head or eye-hand coordination. It is considered that eye and head movement is related to USN symptoms.

In conclusion, it is known that the use of the HMD system according to this embodiment for USN evaluation permits to make clear a left neglect area, which can not be easily viewed in the clinical USN evaluation. Moreover, it may be considered that use of HMD in USN study permits to display an image with a higher accuracy and assess the occurrence and degree of USN with a higher reliability than the common clinical study. HMD can produce various artificial environments which are impossible in the common clinical evaluation.

In the foregoing, the present invention has been described in detail concerning one embodiment thereof as an example. However, it should be understood by those ordinarily skilled in the art that the present invention is not limited to the embodiments but can be modified in various manners, constructed alternatively or embodied in various other forms without departing from the scope and spirit thereof as set forth.

For example, the head mounted display 11 having been explained in the embodiment is just an example and may be constructed in any other form.

As having been described in the foregoing, according to the present invention, since the head mounted display is adapted so that the coordinates in which an image is to be displayed in the head mounted display can be set object- or body-centered, it is more advantageous in many respects than ever as will be described below:

An image may be displayed in object- or body-centered (egocentric) coordinates in the head mounted display for viewing and determination by the examiner, whereby his or her condition can be examined quantitatively. For example, it is possible to assure a new test on the visual space of the examinee. More specifically, the examiner can freely move the coordinate axes. Namely, the examiner can move the coordinate axes to a neglect region of the examinee, to a visible region of the examinee, to an object or to any other region. Also, the examiner can detect the eye movement to know the trajectory of glance of the examinee and hence how an object appears to the examinee looking it. To know how an object appears to an examinee with visual field defect, the examiner can simulate, as an image, how the visual field defect exists in an actual scene. Based on the results of this test, the examiner can give a suitable rehabilitation or vision to each examinee.

Also, the user of the head mounted display according to the present invention can be given an image having added thereto, or modified with, a variety of visual information produced by the computer graphic (CG) technique in such a form that the image is fused with the real world without any border between them. Thus, the examinee can be examined and rehabilitated in a certain limited circumstance as well as in an environment more approximate to the daily life. Therefore, the disorder state of the examinee can be known more easily, multilaterally and accurately, so that the examinee can be examined and rehabilitated efficiently and effectively. Further, since the examination and rehabilitation can be effected with the one and same system, the examinee can be rehabilitated easily in consistency with the results of examination.

Especially, combination of the attention information given to the USN patient with a real-space image greatly helps the examinee recognize the neglect space in the real space.

The present invention is suitably usable with, for example, visual-spatial neglect patients such as USN patients having suffered from a stroke, Parkinson's disease patients, red-green blinded patients, hemianopia patients, low-vision patients, diplopia patients, squinting-eyed patients, elderly persons (normal and perceptional disturbance), eyeglass wearing persons, etc. For example, it is possible to display visual information in other than a visual field defect region of the patients with visual-spatial neglect, diabetic retinopathy, cataract or the like correspondingly to the degree of the visual field defect. For the Parkinson's disease patients, it is possible to improve their walking performance by enhancing the visual information. For example, by enhancing the image of an object, building or the like, it is possible to prevent the patient from overturning, improve the patient's performance of ascending stairs. For the red-green blinded patients, it is possible to display red and green in more-easily discernible colors or the like, respectively.

More particularly, giving visual-information stimuli to the Parkinson's disease patient or the like permits to help him to walk more stably. For example, with a line or bar in a highly distinctive color such as red or yellow being provided on the floor or stairs, the patient tries to go over the line or bar and will hence be able to walk with an increased stride. Thus, by making the edge or color enhancement with the head mounted display, it is possible to always increase the walking stride. The degree of the edge or color enhancement is measured, analyzed and determined with this system to assure a suitable enhancement for each patient.

The patients with cerebellar impediment suffers from nystagmus or the like as an eye movement impediment. Feeling dizzy, they cannot walk stably. Therefore, the movement of eyeballs during nystagmus is measured by the eye movement measuring feature to display an image with the nystagmus being corrected as in the image stabilization. It is thus possible to alleviate the dizziness, movement disorder of upper and lower extremities and gait disorder due to such nystagmus.

For a patient deficient in a visual-field periphery or the like due to diabetic retinopathy or the like, information on the visual field defect captured by the image pickup devices can be displayed, in a reduced scale, in a position around the center of the visual field of the patient. With this system, the amount of the visual-field information on the visual-field defective area is checked and a reduction ratio for the information display is determined based on the information amount.

For a patient having an invisible part in this visual field due to bitemporal hemianopia, binasal hemianopia or the like, visual-field information on the temporal or nasal sides is displayed as a reduced image in a visible area. With this system, the amount of the visual-field information on the visual field difficult for the patient to recognize is checked and a reduction ratio for the information display is determined based on the information amount.

For a patient who cannot distinguish between colors, for example, red and green, this system can be used to provide peculiar patterns to red and green, respectively (vertical lines to red and horizontal lines to green, for example).

Using this system, the amount of visual information on a region of which the color is difficult to recognize is checked and a method necessary for color distinction is determined based on the visual-information amount.

Supplied with right- and left-eye visual information, the head mounted display can produce a spatial perceptual disturbance under conditions more approximate to those in a three-dimensional space. Thus, the spatial perceptual disturbance can be studied accurately and in detail and the patient can be rehabilitated based on the results of study.

1. An examination system comprising a head mounted display, the head-mounted display further comprising:
one or more image pickup devices; and
at least one display unit to display an image to an examinee after being captured by the image pickup device and processed in a pre-determined manner by the processing/control unit, wherein coordinates in which the image is to be displayed in the head mounted display can be set to one of object-centered and body-centered.

2. The examination system according to claim 1, wherein the image pickup device is 3CCD.

3. The examination system according to claim 1, further comprising a digital camera fixed to a chair on which the examinee going to undergo an examination is sitting, the digital camera to capture the image of a test sheet to be formed for display in the head mounted display.

4. The examination system according to claim 3, wherein the processing/control unit is operable to subject the image of the test sheet captured by the digital camera to enlargement or reduction for display in the head mounted display.

5. The examination system according to claim 1, further comprising a motion measuring unit to measure movement of the examinee's head, eye or body during an examination.

6. The examination system according to claim 1, further comprising the processing/control unit.

7-8. (canceled)

9. A method of displaying an image in a head mounted display further comprising:
capturing the image using an image capture device in the head mounted display;
processing the image in a predetermined manner based on a user having a visual defect; and
displaying the image in the head mounted display to the user using one of object-centered and body-centered coordinates.

10. The method of claim 9 wherein the processing further comprises using a selected process to display the image in a manner selected from a group consisting of an edge-enhancing display, a reducing display, an attention information display, and an enlarging display.

11. The method of claim 9 further comprising measuring the movement of the user's head, eye or body.

12. Apparatus for displaying an image in a head mounted display further comprising:
means for capturing the image in the head mounted display;
means for processing the image in a pre-determined manner based on a user having a visual disorder; and
means for displaying the image in the head mounted display to the user using one of object-centered and body-centered coordinates.

13. The apparatus of claim 12 further comprising means for measuring the movement of the user's head, eye or body.

14. An examination system comprising a head mounted display, the head-mounted display further comprising:
one or more image pickup devices; and
at least one display unit to display an image to a user after being captured by the image pickup device and processed in a pre-determined manner by the processing/control unit based on a user having a visual disorder.

15. The examination system according to claim 14, wherein the image pickup device is 3CCD.

16. The examination system according to claim 14 wherein the processing/control unit is operable to process the image for display in a manner selected from a group consisting of an edge-enhancing display, a reducing display, an attention information display, and an enlarging display.

17. The examination system according to claim 14 further comprising a motion measuring unit to measure movement of the user's head, eye or body during an examination.

18. The examination system according to claim 17, further comprising the processing/control unit.
